(19) United States
(12) Patent Application Publication
(10) Pub. No.: US 2007/0053848 A1
(43) Pub. Date: Mar. 8, 2007

(54) Antimicrobial materials for dental care applications

(76) Inventor: Richard F. Stockel, Bridgewater, NJ (US)

Correspondence Address: Law Offices of Jack Matalon, 32 Shelley Rd., Springfield, NJ, 07081-2529, US

(21) Appl. No.: 11/599,758

(22) Filed: Nov. 15, 2006

Related U.S. Application Data
(63) Continuation-in-part of application No. 10/647,752, filed on Aug. 26, 2003, now abandoned.

(51) Int. Cl. A61K 8/81

(52) U.S. Cl. 424/49; 523/115

(57) ABSTRACT

The invention pertains to antimicrobial dental care materials. In particular, the invention pertains to antimicrobial dental care materials containing biocidal complexes, e.g.: a) a mouthwash containing said complex; b) a dentifrice containing said complex; c) a dental floss coated and/or impregnated with said complex; and d) a protective coating for teeth containing said complex, e) toothbrush bristles coated and/or impregnated with said complex; f) an orthodontic appliance coated and/or impregnated with said complex; g) an orthodontic appliance adhesive containing said complex; h) a denture appliance coated and/or impregnated with said complex; I) a denture appliance adhesive containing said complex; j) an endodontic composition coated and/or impregnated with said complex; k) a composite-type dental restorative composition containing said complex; l) a dental cement containing said complex; m) a dental liner containing said complex; n) a dental bonding agent containing said complex; The complex will have a maximum water solubility of about 5 wt. % and is further characterized as being a complex that is formed by a metathesis reaction between a biocidal cationic monomer or polymer with a biocidal anionic monomer or polymer or by an acid-base reaction between a biocidal monomeric or polymeric free base and a biocidal monomeric or polymeric acid capable of protonating the free base.

### REFERENCE TO RELATED APPLICATIONS

This application is a continuation-in-part of application Ser. No. 10/647,752 filed Aug. 26, 2003, the disclosure of which is incorporated herein in its entirety.

### FIELD OF THE INVENTION

This invention relates to antimicrobial dental care materials, e.g., mouthwashes, dentifrices, dental flosses, toothbrush bristles, protective dental coatings, orthodontic appliances and adhesives, denture appliances and adhesives, restorative materials, endodontic materials, an d the like, containing unique complexes of biocides.

### BACKGROUND OF THE INVENTION

The prevention and control of periodontal diseases are important, not only to maintain a healthy and functional natural dentition, but also to reduce the risks of systemic complications.

It is well known that bacteria and their products initiate and perpetuate the process of tissue destruction. Thus, preventive dental care should focus on the bacteria to control periodontal diseases.

Mechanical measures do not appear to maintain periodontal health. Therefore, dental research has been focusing on providing therapeutic agents that will provide better levels of bacteria control. Gingivitis is a rather nonspecific infection. Therefore, desirable anti-plaque agents employed to improve gingival health should have a broad spectrum of antibacterial activity and remain substantive in the oral cavity (i.e., teeth and tissue) for a prolonged period of time.

### OBJECTS OF THE INVENTION

It is an object of the invention to provide a safe and efficacious composition that will have a broad spectrum of activity against bacteria that are responsible for periodontal disease.

It is a further object of the invention to provide a safe and efficacious biocidal composition that will have slow-release properties to insure that its antimicrobial activity will persist in the oral cavity for extended periods of time.

It is an additional object of the invention to provide a wide range of dental care materials that utilize the biocidal composition.

### DETAILED DESCRIPTION OF THE INVENTION

The foregoing objects of the invention and additional objects have been met by providing dental care materials that contain complexes of biocidal components. Broadly speaking, the present invention is directed to a dental care material that may be:
a) a mouthwash containing a complex;
b) a dentifrice containing said complex;
c) a dental floss coated and/or impregnated with said complex; and
d) a protective coating for teeth containing said complex,
e) toothbrush bristles coated and/or impregnated with said complex;
f) an orthodontic appliance coated and/or impregnated with said complex;
g) an orthodontic appliance adhesive containing said complex;
h) a denture appliance coated and/or impregnated with said complex;
I) a denture appliance adhesive containing said complex;
j) an endodontic composition coated and/or impregnated with said complex;
k) a composite-type dental restorative composition containing said complex;
l) a dental cement containing said complex;
m) a dental liner containing said complex;
n) a dental bonding agent containing said complex.

### The Complexes

The complexes employed in the materials of the invention will have a maximum water solubility of about 5 wt. %, preferably 2 wt. %. The complexes are further characterized as having been formed by a metathesis reaction between a biocidal cationic monomer or polymer with a biocidal anionic monomer or polymer or by an acid-base reaction between a biocidal monomeric or polymeric free base and a biocidal monomeric or polymeric acid capable of protanating the free base. The complexes have been found to be extremely effective against a wide variety of microorganisms, e.g., bacteria and fungi. Moreover, the complexes have important safety, efficacy and toxicity implications since the biocidal components employed in the preparation of the complexes by either a metathesis or an acid-base reaction are typically those that have been approved for use by the EPA or the FDA.

The complexes tend to have low water solubility and therefore have prolonged extended-release properties in the oral cavity. For many, but most certainly not all, applications, it is desirable to utilize emulsions or microemulsions of the complexes in order to obtain stable aqueous compositions.

If the biocidal cationic species is utilized in its polymeric form for reaction with the biocidal anionic monomer or polymer, it is preferred that the polymer contains a functionality such as amidine; guanidine; biguanide and quaternary amine; the functionality will be present in the backbone and/or side chains and/or dendrimers of the polymer.

Preferably, both the biocidal cationic species and the biocidal anionic species are utilized in their monomeric form. Suitable biocidal cationic species employed in preparing the complexes by a metathesis reaction will typically contain a functionality such as amidine; guanidine; biguanide; amine-acid salt of an antibiotic; amine-acid salt of an azole; and a quaternary amine.

Exemplary biocidal cationic monomers include a chlorhexidine salt; a cetyl pyridinium halide; a benzalkonium halide, e.g., benzalkonium chloride; a sangiunarine halide, a d,l-pyrrolidone carboxylic acid salt of N-α-cocoyl-1-arginine ethyl ether, domiphen bromide, an ethandiyl-α, ω-bis(dodecyldimethyl) ammonium halide; a delmopinol halide; a tetracycline hydrochloride; a doxycycline hydrochloride; a minocycline hydrochloride or a hydrohalide of ketoconazole, miconazole or tebuconazole.

Other suitable monomeric and polymeric cationic biocides include polyhexamethylene biguanide hydrochloride salt; polyhexamethylene guanidine hydrochloride salt; dimethyldidecyl ammonium chloride; benzethonium chloride; polyionenes, e.g., poly(dimethylbutenyl chloride); α,ω-bis(triethanolammonium chloride); poly[oxyethylene (dimethylimino) ethylene dichloride]; dequalinium chloride; polyquaternium 2; hexetidine; octenidine; tetrakis-(hydroxymethyl)phosphonium sulfate; gemini quats; quaternary ammonium dendrimeric biocides (see U.S. Pat. No. 6,440,405); long chain sulfonium salts; long chain phosphonium salts; and alexidine.

It should be understood that the biocidal monomeric and polymeric cationic species may be present in the form of salts other than a hydrochloride. Other suitable examples include hydrobromide, hydroxy-carboxylic acids, amino acids, sulfates, sulfonates and phosphates.

Suitable biocidal anionic monomers employed in preparing the complexes by a metathesis reaction will typically contain a functionality selected from the group consisting of phenolic; carboxylate; enol; dienol; organophosphate; organophosphinate; organophosphonate; bis-phosphonate; and inorganic phosphorus. Exemplary biocidal anionic monomers include triclosan, o-phenylphenol; thymol; eugenol; tropolone; 4-isopropyl-tropolone; undecylenic acid; mupirocin; a monoalkylphosphate; a dialkylphosphate; ethylenediamino-tetrakis(methylenephosphonic acid); an inorganic phosphate; and an inorganic pyrophosphate. Another class of phenolics useful in the practice of this invention involves natural or synthetic antioxidants.

The following monomeric and polymeric anionic species represent a partial list of suitable biocides that can be combined with the monomeric and polymeric cationic biocides to form the complexes by a metathesis reaction: the alkali metal (e.g., sodium) salts of triclosan, o-phenylphenol; thymol; eugenol; 4-isoppropyltropolone; stearic acid; undecylenic acid; mupirocin; a monoalkylphosphate; a dialkylphosphate; ethylenediaminotetrakis (methylene-phosphonic acid); an inorganic phosphate; and an inorganic pyrophosphate.

Other suitable biocidal monomeric and polymeric anionic species include the alkali metal (e.g., sodium) salts of: hydroxymethyl glycinate; salicylanilide; hinokitiol; poly-phosphate; poly-anionic compositions such as polydivinyl ether-maleic anhydride alternating copolymers; anionic dendrimers such as those disclosed in U.S. Pat. No. 6,464,971; chitosan derivatives having carboxylate, sulfate, sulfonate, phosphonate or phosphate anionic functional groups present in the molecule; EDTA and derivatives thereof containing carboxylic anions; 1-hydroxy-ethane-1,1-diphosphonic acid, nitrilotris(methylenephosphonic acid); amino-phosphonic acids; and antibiotics containing carboxylic acid moieties.

The specific cationic and anionic biocide species set forth above are illustrative of those that can be used to prepare the complexes, but most certainly do not represent a complete inventory of all possible species. Those skilled in the art of chemistry and biology can readily conceptualize other modifications. In particular, some of the biocidal polymeric cationic and anionic species could be further modified by varying the repeating units or by end-capping. U.S. Pat. Nos. 4,891,423 and 5,741,886 describe examples for further enhancement of the-antimicrobial activity of polyhexamethylene-biguanide.

### The Metathesis Reaction

As noted in the McGraw-Hill Dictionary of Scientific and Technical Terms (5^{th} Edition, 1994), metathesis is a reaction involving the exchange of elements or groups as in the general reaction: AX+BY→AY+BX.

The metathesis reaction is straight forward and can be readily carried out in aqueous solutions using water alone or a mixture of water and up to about 85 wt. % of a solvent such as a C₁-C₄ alcohol, e.g., methanol, ethanol, isopropanol, n-butanol, etc. Typically the water alone or water-alcohol solvent will be utilized in an amount of about 40 to about 85 wt. %, based on the weight of the reaction mixture.

An alkali or alkaline earth metal (e.g., Na, K, Li, Ca, etc.) salt of the selected biocidal anionic monomer or polymer is formed by reacting it with an equivalent amount of an alkali or alkaline earth metal hydroxide in water or water-alcohol solution. An acid salt, e.g., acetate, hydrohalide, gluconate, sulfate, etc. of the selected free base biocidal monomer or polymer is formed by reacting it with an equivalent amount of an acid such as acetic, hydrochloric, hydrobromic, gluconic acid, sulfuric, etc. in water or water-alcohol solution.

Thereafter, an equivalent amount of the aqueous alkali or alkaline earth metal salt solution of the selected biocidal anionic monomer or polymer is mixed with the aqueous acid salt solution of the selected cationic monomer or polymer. The concentration of the reactants can vary from about 20 wt. % to about 60 wt. % of the total reaction mixture. Mixing is continued at room temperature for several minutes up to about one hour. The reaction product may be readily recovered by decantation of the supernatant layer (which contains the byproduct salts) or by filtration. The solid layer consisting of the complex may be used as is for many of the materials recited above or dried (e.g., in air, in vacuuo at a temperature of about 50 to about 130° C., etc.). If desired, the complex may be recrystallized using a solvent such that the solubility of the complex in the solvent is low at room temperature, but the solubility increases significantly near the boiling point of the solvent.

### The Acid-Base Reaction

As mentioned above, it is preferred to use an acid-base reaction to prepare the desired complex if the selected biocidal monomeric or polymeric acid is capable of protonating the selected monomeric or polymeric free base. The use of the acid-base reaction avoids the necessity of forming an alkali metal salt of the selected biocidal anionic monomer or polymer and the acid salt of the selected biocidal cationic monomer or polymer and having to dispose of the salt byproduct.

Biocidal primary, secondary and/or tertiary amines which form salts with acids and are capable of being protonated may be utilized to form useful biocidal complexes which are employed in the dental care materials of the invention.

Preferably, the free base comprises a tertiary amine selected from the group consisting of sanguinarine, tetracycline; doxycycline; minocycline; and delmopinol. The biocidal monomeric acid capable of protonating the free base will typically be a carboxylic acid such as undecylenic acid, stearic acid, mupirocin or salicyclic acid.

The acid-base reaction of a conjugate base (i.e., the free base) of the selected biocidal cationic monomer or polymer with the conjugate acid (protonated) of the selected biocidal anionic monomer or polymer may be illustrated by the following example:
chlorhexidine+undecylenic acid→chlorhexidinium undecylenate complex base acid

In order for the acid-base reaction to proceed, the acid component must have a transferable proton (Pₖₐ) to a basic (P_{kb}) molecule. The acid-base reaction is usually conducted in refluxing alcohol (e.g., a C₁-C₄ alcohol such as methanol, ethanol, isopropanol, n-butanol, etc.) or aqueous alcoholic solution (e.g., about 10 to about 90 wt. % water) and the reaction is typically complete in one hour or less. The complex may be readily recovered from the reaction mixture by filtration, air drying, removal of the solvent in vacuuo at a temperature of about 50 to about 130° C., etc. If desired, the complex may be recrystallized using a solvent such that the solubility of the complex in the solvent is low at room temperature, but the solubility increases significantly near the boiling point of the solvent.

The acid-base reaction is particularly advantageous for the formation of biocidal azole complexes of biocides that have a protonic hydrogen capable of transfer to a base nitrogen in an azole molecule. The azoles are either imidazole or triazole derivatives. If the azole can be protonated, then it can be subsequently reacted with an anionic monomeric or polymeric biocide.

It is clear that the complexes employed in the dental care materials of the invention are not mere admixtures of the biocidal anionic monomer or polymer and the biocidal cationic monomer or polymer, but rather they are different compositions. For example, chlorhexidine gluconate may be reacted (by the metathesis reaction) with thymol in the presence of an aqueous solution of sodium hydroxide (thereby forming the sodium salt of thymol). The chlorhexidinium dithymolate complex resulting from the reaction has a sharp melting point of 123-124.5° C. In contradistinction, the chlorhexidine gluconate acid salt decomposes rather than melts, the chlorhexidine free base has a melting point of 134-136° C., and thymol has a melting point of 51.5° C. Moreover, as shown in Example 3 set forth below, the complex provides a slow biocidal release over a period of time whereas chlorhexidine and thymol individually have relatively little residual biocidal activity.

### Formation of Emulsions/Microemulsions of the Complexes

As mentioned above, the complexes employed in the dental care materials of the invention typically have limited water solubility. Therefore, for many dental care materials, e.g., mouthwashes and dentifrices, it is desirable to utilize the complexes in the form of emulsions or microemulsions. The following is a generalized procedure for preparing emulsions or microemulsions of the complexes.

First, the complex is dissolved in the minimum amount of a solvent that will completely dissolve the selected complex in the amount that is intended for use in the desired dental care material. The solvent of choice will be one with the appropriate Hildebrand solubility parameter. The solubility parameter is a numerical value that indicates the relative solvency behavior of a specific solvent Hildebrand solubility parameters of about 8.5 to about 22.0 are generally suitable for solubilization of the complexes. Exemplary solvents with the requisite Hildebrand solubility parameters include ethanol, glycerine, propylene glycol, sorbitol, methanol and the like.

The desirable Hildebrand solubility parameter will depend on the ionic/covalent bonding energies of the complexes. The correct solvent will be one having a relatively low Hildebrand solubility parameter if the bonding has more covalency and a relatively high Hildebrand solubility parameter if the bonding is more ionic. Of course, combinations of correct solvents may also be utilized to dissolve the complexes.

Thereafter, a surfactant is added to the dissolved complex. The surfactant may be cationic, anionic or amphoteric in nature, and combinations of the different types or combinations of the same type of surfactants may be use. Preferably, the surfactant will be amphoteric or nonionic in nature. Highly negative anionic surfactants are not very functional.

The last step is to dilute the complex-solvent-surfactant composition with water to the concentration desired for the selected dental care material so as to form an emulsion or microemulsion depending on the micellar size and the choice of solvents/cosolvents.

### The Surfactants

For the purposes of this invention, it is preferred that the surfactants employed in the formation of microemulsions (cosolvents are added) or emulsions of the complexes are generally of the nonionic or amphoteric type or combinations of one or more nonionics, one or more amphoterics or one or more nonionics in combination with one or more amphoterics. Highly charged anionic surfactants are less desirable since they have the potential to reduce the biocidal activity of the complexes by causing some degree of precipitation, thereby lessening the effectiveness of the complexes.

It has also been found that cationic phospholipids, preferably in combination with nonionic and/or amphoteric surfactants are effective in the formation of microemulsions or emulsions of the complexes.

Surfactants that carry a positive charge in strongly acidic media carry a negative charge in strongly basic media, and form zwitterionic species at intermediate pH levels are amphoteric. The preferred pH range for stability and effectiveness is about 5.0 to about 9.0. Within this pH range, the amphoteric surfactant is mostly or fully in the zwitter (neutral) form, thereby negating any dilution of biocidal activity of the complexes, provided that the surfactant is employed in the preferred concentration range of about 0.25 to about 4.0 wt. %, based on the weight of the complex in the final formulation.

The following surfactants have been found to be effective in the formation of microemulsions or semitransparent emulsions of the complexes: amphoteric amidobetaines; nonionic polyethoxylated sorbital esters, polycondensates of ethylene oxide-propylene oxides (polyoxamers), polyethoxylated hydrogenated castor oils, and certain cationic phospholipids.

Suitable examples of amidobetaines include cocoamidoethyl betaine, cocoamidopropyl betaine; and mixtures thereof. Other suitable amphoteric surfactants include long chain imidazole derivatives such as the product marketed under the trade name “Miranol C2M” by Rhodia and long chain betaines such as the product marketed under the trade name “Empigen BB” by Huntsman Corporation, and mixtures thereof.

Suitable nonionic surfactants include polyethoxylated sorbitol esters, especially polyethoxylated sorbital monoesters, e.g., PEG sorbitan di-isostearate, and the products marketed under the trade name “Tween” by ICI; polycondensates of ethylene oxide and propylene oxide (polyoxamers), e.g., the products marketed under the trade name “Pluronic” by BASF; condensates of propylene glycol; polyethoxylated hydrogenated castor oil such as the products marketed under the trade name “Cremophors” by BASF; and sorbitan fatty esters marketed by ICI. Other effective nonionic surfactants include the polyalkyl (C₈-C₁₈) glucosides.

Suitable cationic surfactants include D,L-pyrrolidone-5-carboxylic acid salt of ethyl-cocoyl-L-arginate (CAE) marketed by Ajinomoto, and cocoamidopropyl (PTC), lauramidopropyl PG diammonium chloride phosphates and the like marketed by Uniqema. CAE and PTC have significant biocidal activity and they therefore can be used as the cation of the binary cationic-anionic biocidal complexes.

### Mouthwash

The biocidal complexes are especially useful for the formulation of mouthwashes. Such mouthwashes will typically comprise the following components:
a) about 0.01 to about 1.5 wt. %, based on the weight of the mouthwash, of a monomeric or polymeric biocidal complex formed by the metathesis reaction or by the acid-base reaction as described above;
b) about 0.25 to about 4.0 wt. %, based on the weight of the mouthwash, of an orally acceptable cationic, anionic, or amphoteric surfactant or mixtures of such surfactants;
c) 0 to about 20 wt. % of ethanol; and
d) the balance of the mouthwash comprising water.

For the purposes of the present invention, the term “orally acceptable” means that the selected component, e.g., surfactant, thickening polymer, humectant, solvent, conformal polymer, etc. will be safe and efficacious in the oral cavity. Of course, the selected component must not have any adverse effect on the biocidal activity of the selected complex.

The amphoteric amidobetaine surfactants are particularly useful in formulating clear, aqueous or aqueous-alcohol mouthwash formulations.

In addition to the components set forth above, the mouthwash formulation may contain the usual incipients found in mouthwashes, e.g., liquids such as glycerin or propylene glycol, humectants, thickening agents, chelating agents, organic carboxylic acids, flavoring agents, sweetening agents, coloring agents, preservatives, etc.

### Dentifrice

The complexes are quite useful in the formulation of a dentifrice for reducing the formation of plaque, thereby inhibiting periodontal disease.

Dental plaque is a soft deposit which forms on teeth and is comprised of an accumulation of bacteria and bacterial by-products. Dental plaque adheres tenaciously at the points of irregularity or discontinuity, e.g., on rough calculus surfaces, at the gum line and the like. Besides being unsightly, plaque is implicated in the occurrence of gingivitis and other forms of periodontal disease.

Chlorhexidine and triclosan are perhaps the best-known antiplaque agents; they have been investigated by numerous scientists and they are widely used in formulating dentifrices available on the current market. Chlorhexidine is acknowledged to be more effective than triclosan in combating plaque. However, chlorhexidine causes noticeable staining of the teeth for the majority of users. This unsightly stain can only be removed in the course of a dental office visit where it is removed by mechanical means. Attempts to include abrasives and anionic surfactants in the chlorhexidine-containing dentifrice to reduce staining have not proven to be successful due to the incompatibility of the chlorhexidine with such materials, and thereby resulting in a diminution of the biocidal activity of the chlorhexidine.

The biocidal complexes can be readily formulated into dentifrices having effective anti-plaque properties with little or no staining accompanying their use. Such staining typically comes from a cationic biocide, e.g., chlorhexidine, cetyl pyridinium chloride, quats, etc., which exist in a water-soluble form in the oral cavity. Furthermore, the complexes have limited water solubility and therefore dentifrices containing the complexes probably operate as a slow-release reservoir of the complex.

The dentifrice compositions of the invention will generally comprise the following components:
a) about 0.01 to about 5.0 wt. %, based on the weight of the dentifrice, of a monomeric or polymeric biocidal complex formed by the metathesis reaction or by the acid-base reaction as described above;
b) about 0.1 to about 5.0 wt. %, based on the weight of the dentifrice, of an orally acceptable cationic, anionic, or amphoteric surfactant;
c) 0 to about 5.0 wt., based on the weight of the dentifrice, of an orally acceptable thickening polymer;
d) 0 to about 15 wt. %, based on the weight of the dentifrice, of an orally acceptable humectant;
e) about 5.0 to about 20.0 wt. %, based on the weight of the dentifrice, of an orally acceptable solvent; and
f) the balance being water (preferably deionized water).

Suitable humectants include sorbitol, glycerin, glycols, and the like. Suitable thickening polymers include hydrocolloids, acrylates, acrylamides and the like. Suitable solvents include ethanol, isopropanol, propylene glycol, sorbital and the like. The dentifrices of the invention may also contain the other incipients that are conventionally present in current dentifrices, e.g., colorants, flavorants, sweeteners, abrasives, thickeners, foaming agents, etc. A typical dentifrice formulation employing a complex is set forth in Table 1.

TABLE 1

| Ingredient | % by Weight |
|---|---|
| Complex | 0.5 |
| Glycerin | 8.0 |
| Sodium carboxymethyl cellulose | 1.5 |
| Sorbital | 38.0 |
| Sodium monofluorophosphate | 0.8 |
| Saccharin, sodium | 1.0 |
| Sodium dihydrogen phosphate | 0.25 |
| Sodium monohydrogen phosphate | 0.25 |
| Silica, hydrated | 15.0 |
| Titanium dioxide | 0.25 |
| Flavoring agent | 2.0 |
| FD & C dye | 0.0003 |
| Deionized water | Q.S. to 100 |

### Dental Floss

An important use for the complexes involves biocidal dental floss. It is well known that periodontal disease affects the supporting tissues of teeth, bone, periodontal ligament, cementum and gingival. As is well known, periodontal disease is caused by bacterial plaque formation on teeth surfaces. The most difficult areas to reach by brushing or mouthwash for proper oral hygiene are the interproximal surfaces of the teeth. These areas are best cleaned with the aid of dental floss. However, the various types of dental flosses disclosed in the prior art typically effect only a mechanical cleaning of the interproximal teeth areas.

Dental flosses have long been used effectively to clean the spaces between the teeth and under the gum margin. To increase the effectiveness of the floss, fluoride or bactericides may be added to the floss in bulk or as a coating. The proper use of a dental floss has been found to be effective in inhibiting tooth decay and gum diseases.

Dental flosses can be made of natural or synthetic fibers, e.g., teflon, nylon, polypropylene, etc., and it can contain a wax to reduce friction.

The complexes can either be dispersed or dissolved in commonly used binders, e.g., wax, hydrophilic polymers, polyalkylene glycols, etc., to coat and/or impregnate the dental floss material. Certain complexes wherein the anionic moiety is a long-chain carboxylate can function as an anti-friction agent which retaining the biocidal activity of the complex.

Typically, the coated or impregnated dental floss will contain the biocidal complex in an amount of about 0.10 to about 10 wt. %, based on the weight of the floss. The biocidal complexes will slowly erode off the dental floss and deposit on the tooth structure and the gums when used to clean the teeth. The following example describes how a non-waxed commercial dental floss can be coated with a chlorhexidine-triclosan complex for use as a germicidal dental floss. A suitable dental floss is set forth below in Example 1.

### Protective Coating

An important use of the biocidal complexes involves a protective coating for the teeth which may be painted onto the teeth to provide long-term protection against caries. Typically, the protective coating will comprise:
a) about 1.0 to about 15 wt. %, based on the weight of the coating, of the complex;
b) about 5 to about 30 wt. % of an orally acceptable conformal polymer; and
c) the balance being an orally acceptable solvent.

Suitable orally acceptable conformal polymers include polypropylene glycol, poly-vinyl acetate-vinyl alcohol, poly-2-hydroxyethyl methacrylate and the like. Other polymers may also be used, provided they possess slight water solubility, are orally acceptable (i.e., they are safe and efficacious) and are compatible with an orally acceptable solvent such as ethanol, isopropanol, propylene glycol and the like. Particularly useful complexes are chlorhexidine-triclosan, chlorhexidine-thymol, polyhexamethylene biguanide-triclosan and polyhexamethylene biguanide-thymol. A typical formulation for a protective coating is shown in Example 2 set forth below.

The following nonlimiting examples shall serve to illustrate the various embodiments of this invention. Unless otherwise indicated, all parts and percentages are on a weight basis.

### EXAMPLE 1

To a 5 g sample of a chlorhexidine-triclosan complex was added 60 g of PEG 3350, 30 g PEG 1000 and 5 g glycerin. The mixture was gently heated and stirred to dissolve the complex. The resultant warm solution was used to coat a commercial non-wax dental floss to provide an efficacious germicidal dental floss.

### EXAMPLE 2

| | |
|---|---|
| chlorhexidine-triclosan complex | 5% w/w |
| 60% vinyl acetate-40% vinyl alcohol copolymer | 20% w/w |
| ethanol | 75% w/w |

The following example illustrates the long-term advantages of the biocidal complexes:

### EXAMPLE 3

A microemulsion containing 1.0 wt. % of a complex consisting of didodecyldimethyl ammonium chloride and the sodium salt of triclosan was formulated using propylene glycol and “Tego Betaine-ZF” as the amphoteric surfactant.

A 50 ml portion of the microemulsion was inoculated with a 0.5 ml suspension of Escherichia coli (the initial microorganism count was 10⁸ cfu/ml) and stirred. The resultant solution was then streaked onto triple agar plates containing tryptone soya agar and the plates were then incubated at 37° C. for 48 hours. Thereafter, the plates were inspected at 3, 6, 24, 48, 72 and 168 hours. No microbial growth was observed in any of the three plates at any of the indicated hours.

What is claimed is:

1. An antimicrobial dental care material containing a biocidal complex, said material being selected from the group consisting of:
a) a mouthwash containing said complex;
b) a dentifrice containing said complex;
c) a dental floss coated and/or impregnated with said complex; and
d) a protective coating for teeth containing said complex,
e) toothbrush bristles coated and/or impregnated with said complex;
f) an orthodontic appliance coated and/or impregnated with said complex;
g) an orthodontic appliance adhesive containing said complex;
h) a denture appliance coated and/or impregnated with said complex;
I) a denture appliance adhesive containing said complex;
j) an endodontic composition coated and/or impregnated with said complex;
k) a composite-type dental restorative composition containing said complex;
l) a dental cement containing said complex;
m) a dental liner containing said complex;
n) a dental bonding agent containing said complex;
said biocidal complex being characterized as having a maximum water solubility of about 5 wt. % and further characterized as being a biocidal complex that is formed by a metathesis reaction between a biocidal cationic monomer or polymer with a biocidal anionic monomer or polymer or by an acid-base reaction between a biocidal monomeric or polymeric free base and a biocidal monomeric or polymeric acid capable of protonating the free base.

2. The material of claim 1 wherein said complex has a maximum water solubility of 2 wt. %.

3. The material of claim 1 wherein said complex is formed by a metathesis reaction between a biocidal cationic polymer and a biocidal anionic monomer or polymer, said cationic polymer containing a functionality selected from the group consisting of amidine; guanidine; biguanide and quaternary amine, said functionality being present in the backbone and/or side chains and/or dendrimers of the polymer.

4. The material of claim 1 wherein said complex is formed by a metathesis reaction between a biocidal cationic monomer and a biocidal anionic monomer, said biocidal cationic monomer containing a functionality selected from the group consisting of amidine; guanidine; biguanide; amine-acid salt of an antibiotic; amine-acid salt of an azole; and quaternary amine.

5. The material of claim 4 wherein said biocidal cationic monomer is selected from the group consisting of a chlorhexidine salt; a cetyl pyridinium halide; a benzalkonium halide; a sangiunarine halide, a d,l-pyrrolidone carboxylic acid salt of N-α-cocoyl-1-arginine ethyl ether, domiphen bromide, an ethandiyl-α, ω-bis(dodecyldimethyl) ammonium halide; a delmopinol halide; tetracycline hydrochloride; doxycycline hydrochloride; minocycline hydrochloride; cloconazole; clotrimazole; fenbuconazole; propiconazole; tebuconazole; miconazole; myclobutanil; and ketoconazole.

6. The material of claim 1 wherein said complex is formed by a metathesis reaction between a biocidal cationic monomer and a biocidal anionic monomer, said biocidal anionic monomer containing a functionality selected from the group consisting of phenolic; carboxylate; enol; dienol; organophosphate; organophosphinate, organophosphonate; bis-phosphonate; and inorganic phosphorus.

7. The material of claim 6 wherein said biocidal anionic monomer is selected from the group consisting of the alkali metal salts of triclosan, o-phenylphenol; thymol; eugenol; tropolone; 4-isoppropyltropolone; stearic acid; undecylenic acid; mupirocin; a monoalkylphosphate; a dialkylphosphate; ethylenediaminotetrakis(methylenephosphonic acid); an inorganic phosphate; and an inorganic pyrophosphate.

8. The material of claim 1 wherein said complex is formed by an acid-base reaction between a biocidal monomeric free base and a biocidal monomeric acid capable of protonating the free base.

9. The material of claim 8 wherein said free base comprises a tertiary amine selected from the group consisting of sanguinarine, tetracycline; doxycycline; minocycline; and delmopinol.

10. The material of claim 8 wherein said biocidal monomeric acid comprises a carboxylic acid selected from the group consisting of undecylenic acid; stearic acid; mupirocin; and salicyclic acid.

11. The material of claim 1 present in the form of a mouthwash comprising:
a) about 0.01 to about 1.5 wt. %, based on the weight of the mouthwash, of said complex;
b) about 0.25 to about 4.0 wt. %, based on the weight of the mouthwash, of an orally acceptable cationic, anionic, or amphoteric surfactant or mixtures of such surfactants;
c) 0 to about 20 wt. % of ethanol; and
d) the balance of the mouthwash comprising water.

12. The material of claim 4 present in the form of a mouthwash comprising:
a) about 0.01 to about 1.5 wt. %, based on the weight of the mouthwash, of said complex;
b) about 0.25 to about 4.0 wt. %, based on the weight of the mouthwash, of an orally acceptable cationic, anionic, or amphoteric surfactant or mixtures of such surfactants;
c) 0 to about 20 wt. % of ethanol; and
d) the balance of the mouthwash comprising water.

13. The material of claim 6 present in the form of a mouthwash comprising:
a) about 0.01 to about 1.5 wt. %, based on the weight of the mouthwash, of said complex;
b) about 0.25 to about 4.0 wt. %, based on the weight of the mouthwash, of an orally acceptable cationic, anionic, or amphoteric surfactant or mixtures of such surfactants;
c) 0 to about 20 wt. % of ethanol; and
d) the balance of the mouthwash comprising water.

14. The material of claim 1 present in the form of a dental floss wherein the coated or impregnated complex is present in an amount of about 0.10 to about 10 wt. %, based on the weight of the floss.

15. The material of claim 4 present in the form of a dental floss wherein the coated or impregnated complex is present in an amount of about 0.10 to about 10 wt. %, based on the weight of the floss.

16. The material of claim 6 present in the form of a dental floss wherein the coated or impregnated complex is present in an amount of about 0.10 to about 10 wt. %, based on the weight of the floss.

17. The material of claim 1 in the form of a dentifrice comprising:
a) about 0.01 to about 5.0 wt. %, based on the weight of the dentifrice, of said complex;
b) about 0.1 to about 5.0 wt. %, based on the weight of the dentifrice, of an orally acceptable cationic, anionic, or amphoteric surfactant;
c) 0 to about 5.0 wt., based on the weight of the dentifrice, of an orally acceptable thickening polymer;
d) 0 to about 15 wt. %, based on the weight of the dentifrice, of an orally acceptable humectant;
e) about 5.0 to about 20.0 wt. %, based on the weight of the dentifrice, of an orally acceptable solvent; and
f) the balance of the dentifrice comprising water.

18. The material of claim 4 in the form of a dentifrice comprising:
a) about 0.01 to about 5.0 wt. %, based on the weight of the dentifrice, of said complex;
b) about 0.1 to about 5.0 wt. %, based on the weight of the dentifrice, of an orally acceptable cationic, anionic, or amphoteric surfactant;
c) 0 to about 5.0 wt., based on the weight of the dentifrice, of an orally acceptable thickening polymer;
d) 0 to about 15 wt. %, based on the weight of the dentifrice, of an orally acceptable humectant;
e) about 5.0 to about 20.0 wt. %, based on the weight of the dentifrice, of an orally acceptable solvent; and
f) the balance of the dentifrice comprising water.

19. The material of claim 6 in the form of a dentifrice comprising:
a) about 0.01 to about 5.0 wt. %, based on the weight of the dentifrice, of said complex;
b) about 0.1 to about 5.0 wt. %, based on the weight of the dentifrice, of an orally acceptable cationic, anionic, or amphoteric surfactant;
c) 0 to about 5.0 wt., based on the weight of the dentifrice, of an orally acceptable thickening polymer;
d) 0 to about 15 wt. %, based on the weight of the dentifrice, of an orally acceptable humectant;
e) about 5.0 to about 20.0 wt. %, based on the weight of the dentifrice, of an orally acceptable solvent; and
f) the balance of the dentifrice comprising water.

20. The material of claim 1 in the form of a protective coating for teeth comprising:
a) about 1.0 to about 15 wt. %, based on the weight of the coating, of said complex;
b) about 5 to about 30 wt. %, based on the weight of the coating, of an orally acceptable conformal polymer; and
c) the balance being an orally acceptable solvent.

21. The material of claim 4 in the form of a protective coating for teeth comprising:
a) about 1.0 to about 15 wt. %, based on the weight of the coating, of said complex;
b) about 5 to about 30 wt. %, based on the weight of the coating, of an orally acceptable conformal polymer; and
c) the balance being an orally acceptable solvent.

22. The material of claim 6 in the form of a protective coating for teeth comprising:
a) about 1.0 to about 15 wt. %, based on the weight of the coating, of said complex;
b) about 5 to about 30 wt. %, based on the weight of the coating, of an orally acceptable conformal polymer; and
c) the balance being an orally acceptable solvent.
